(19) United States
(12) Patent Application Publication
(10) Pub. No.: US 2018/0093104 A1
(43) Pub. Date: Apr. 5, 2018

(54) LIGHT IRRADIATION METHOD, LIGHT IRRADIATION DEVICE, LIGHT IRRADIATION SYSTEM, DEVICE SYSTEM FOR PHOTODYNAMIC DIAGNOSIS OR PHOTODYNAMIC THERAPY, SYSTEM FOR SPECIFYING TUMOR SITE AND SYSTEM FOR TREATING TUMOR

(71) Applicant: SONY CORPORATION, TOKYO (JP)

(72) Inventors: KAZUKI IKESHITA, SAITAMA (JP); TAKUYA KISHIMOTO, TOKYO (JP); TAKASHI YAMAGUCHI, KANAGAWA (JP); KOICHIRO KISHIMA, KANAGAWA (JP); HIROSHI MAEDA, KANAGAWA (JP)

(21) Appl. No.: 15/559,939

(22) PCT Filed: Feb. 26, 2016
(86) PCT No.: PCT/JP2016/056691

§ 371 Date: Sep. 20, 2017

(30) Foreign Application Priority Data
Mar. 31, 2015 (JP) 2015-074513

(51) Int. Cl. A61N 5/06; A61B 1/06

(52) U.S. Cl. CPC A61N 5/062; A61N 2005/0612; A61B 1/06

(57) ABSTRACT

The present invention provides a light irradiation method using a pulse laser, which can be applied to technology of PDD and/or PDT, and a system for treating a tumor, which has an enhanced tumoricidal effect. A light irradiation method of irradiating a cell into which a photosensitive compound capable of generating singlet oxygen has been incorporated with a pulse laser having a wavelength within a Soret band is provided. The pulse width of the pulse laser can be set to 100 psec or less, and the wavelength can be set to 405±10 nm. As the photosensitive compound capable of generating singlet oxygen, an agent for photodynamic therapy (PDT) or an agent for photodynamic diagnosis (PDD) can be used.

### TECHNICAL FIELD

The present invention relates to a light irradiation method, a light irradiation device, a light irradiation system, a device system for photodynamic diagnosis or photodynamic therapy, a system for specifying a tumor site, and a system for treating a tumor.

### BACKGROUND ART

Conventionally, in the protocol from diagnosis to surgery of cancer, firstly, screening diagnosis of cancer, and then cytologic diagnosis are performed, and if diagnosed as cancer, preoperative chemotherapy, and then surgery are performed in this order.

For example, with regard to breast cancer, a molecular target drug such as Herceptin has appeared in recent years, and the treatment outcome has improved and the prognosis of a patient has also improved, therefore, chemotherapy has been actively performed. In addition, depending on the case, after chemotherapy, if image diagnosis is performed by computed tomographic (CT) scanning, or the like, a result of cancer disappearance has been also obtained.

However, even if it seems that the cancer area has disappeared by image diagnosis such as CT scanning, it is considered that there are not a few cancer cells remaining in the area. Therefore, chemotherapy, and then further surgery are performed. In the surgery, it is also examined whether or not cancer has remained by examining the resection stump. In a case where the resection stump is determined to be positive, it is determined that re-resection is required for the case, and such a case tends to be increased.

In this background, even if the cancer area contracts by preoperative chemotherapy, there is a type that the cancer part diminishes while remaining in an enclave state (Non Patent Document 1); along with the disappearance of a cancer part, the surrounding fibrosis and inflammation disappear and the whole shape of the breast also changes, and the area where the cancer existed before the preoperative chemotherapy becomes unclear; and further, different from endoscopic surgery and the like, in the conventional breast cancer surgery, a doctor performs the surgery while visually observing the surgical field, therefore, there is a case where it is difficult to find the cancer part remaining in an enclave state in the surroundings by preoperative chemotherapy, and the like.

Surgery after preoperative chemotherapy and the subsequent re-resection affect the prognosis and also the quality of life of a patient after the surgery. Improvement of the surgical method and the like in recent years contributes to the improvement of the prognosis and the quality of life of a patient, but in order to achieve further improvement, a surgical method in which the surgical resection area is reduced so that the appearance of the breast is not changed as compared with the appearance of the breast before the surgery, diagnostic and surgical methods of cancer without using a surgical knife, and the like have been developed.

As the diagnostic and surgical methods of cancer without using a surgical knife, for example, there are photodynamic diagnosis (hereinafter, also referred to as “PDD”) and photodynamic therapy (hereinafter, also referred to as “PDT”).

The photodynamic diagnosis (PDD) has fewer side effects, and is a diagnostic method in which a photosensitive reagent having an affinity specific for a malignant tumor is intravenously or orally administered to a subject so as to be accumulated selectively in a tumor tissue, and then excited by irradiation with light at a specific wavelength, and by observing the fluorescent color, the site of the tumor is specified.

The photodynamic diagnosis (PDT) is a method in which a photosensitive therapeutic drug is intravenously injected so as to be accumulated selectively in a tumor tissue, and then by the irradiation with light at a specific wavelength to excite, singlet oxygen (active oxygen) having a high tumoricidal effect is produced, and only the tumor cells are necrotized for the treatment without causing thermal destruction of the surrounding normal tissue cells.

For example, in Patent Document 1, an electronic endoscope system in which drugs for PDD and PDT are administered into a target site to be treated in a subject, and by the irradiation with light at 405 nm, the target site to be treated is specified by the fluorescence emitted by the drugs, and then by the irradiation with light in the vicinity of 630 nm, the treatment is performed has been disclosed.

### CITATION LIST

### Patent Document

### Patent Document 1: Japanese Patent Application Laid-Open No. 2012-65899

### Non Patent Document

Non Patent Document 1: Rie Horii and Futoshi Akiyama, “Histological assessment of therapeutic response in breast cancer”, Breast cancer (2013)

### SUMMARY OF THE INVENTION

Problems to be Solved by the Invention

However, the technologies of PDD and PDT have been applied to specific cancers such as lung cancer, esophageal cancer, cervical cancer, and brain tumor, but have not been applied yet to breast cancer, and the like.

An object of the present technology is mainly to provide a light irradiation method in which a highly-repeated pulse laser is used and which can be applied to PDD and/or PDT technologies, and a system for treating a tumor having an enhanced tumoricidal effect.

### Solutions To Problems

In order to solve the above problems, the present technology provides a light irradiation method in which irradiation of a cell into which a photosensitive compound capable of generating singlet oxygen has been incorporated with a highly-repeated pulse laser having a wavelength within a Soret band is performed.

The pulse width of the highly-repeated pulse laser can be set to 100 psec or less, and the wavelength can be set to 405±10 nm. The repetition can be 80 Hz or more.

As the photosensitive compound capable of generating singlet oxygen, an agent for photodynamic therapy (PDT) or an agent for photodynamic diagnosis (PDD) can be used.

In addition, the photosensitive compound capable of generating singlet oxygen may be a compound having a cyclic tetrapyrrole.

The compound having a cyclic tetrapyrrole may be a compound generated by metabolism.

In addition, the cyclic tetrapyrrole may be porphyrin.

Further, examples of the cell include a tumor cell, and particularly a breast cancer cell.

In addition, the present technology provides a light irradiation device equipped with a light irradiation unit for irradiating a cell into which a photosensitive compound capable of generating singlet oxygen has been incorporated with a highly-repeated pulse laser having a wavelength within a Soret band.

Further, the present technology provides a device system for photodynamic diagnosis or photodynamic therapy including a light irradiation device equipped with a light irradiation unit for irradiating a cell into which a photosensitive compound capable of generating singlet oxygen has been incorporated with a highly-repeated pulse laser having a wavelength within a Soret band, and

an image acquisition device equipped with a first imaging unit for imaging an image by fluorescence emitted from a cell into which a photosensitive compound capable of generating singlet oxygen has been incorporated.

The image acquisition device can be equipped with a second imaging unit for imaging an image of the cell by natural light.

The present technology provides a system for specifying a tumor site including

an agent containing a photosensitive compound capable of generating singlet oxygen, and

a light irradiation device equipped with a light irradiation unit for irradiating a cell into which the photosensitive compound has been incorporated with a highly-repeated pulse laser having a wavelength within a Soret band.

Furthermore, the present technology provides a system for treating a tumor including

an agent containing a photosensitive compound capable of generating singlet oxygen, and

a light irradiation device equipped with a light irradiation unit for irradiating a cell into which the photosensitive compound has been incorporated with a highly-repeated pulse laser having a wavelength within a Soret band.

Note that, in the present technology, the expression “Soret band” means the light having a wavelength of 300 nm to 600 nm.

In addition, in the present technology, the expression “photosensitive compound capable of generating singlet oxygen” may be originally a photosensitive compound that is excited by light irradiation and generates singlet oxygen, or may be a derivative of the photosensitive compound; or may be a photosensitive compound that is originally not the photosensitive compound capable of generating singlet oxygen but a photosensitive compound capable of generating singlet oxygen by metabolism before being taken into a cell and being irradiated with a pulse laser, and includes all of these compounds.

### Effects of the Invention

According to the present technology, the tumoricidal effect in photodynamic therapy (PDT) can be enhanced, and the present technology can be applied also to photodynamic diagnosis (PDD).

Note that the effects described herein are not necessarily limited and may be any of the effects described in the present disclosure.

### BRIEF DESCRIPTION OF DRAWINGS

FIG. 1 is a graph of absorbance of talaporfin, porfimer and hemoglobin.

FIG. 2 is a schematic diagram showing an arrangement example of components of the device for photodynamic diagnosis or device system for photodynamic therapy according to the present technology.

FIG. 3 is a schematic diagram showing an example of the integrated camera according to the present technology.

FIG. 4 is a schematic diagram showing the first embodiment of the device system for photodynamic diagnosis according to the present technology.

FIG. 5 is a conceptual diagram showing an image taken by the camera for PDD observation/PDT according to the present technology.

FIG. 6 is a conceptual diagram showing a fusion image of the camera for PDD observation/PDT and natural light observation camera according to the present technology.

FIG. 7 is a picture showing a fluorescent image of dish A (irradiated with a pulse laser for 30 minutes without the addition of talaporfin).

FIG. 8 is a picture showing a fluorescent image of dish B (irradiated with a pulse laser for 30 minutes after the lapse of 24 hours from the addition of talaporfin).

FIG. 9 is a picture showing a fluorescent image of dish A (irradiated with a pulse laser for 10 minutes without the addition of talaporfin).

FIG. 10 is a picture showing a fluorescent image of dish B (irradiated with a pulse laser for 10 minutes after the lapse of 24 hours from the addition of talaporfin).

FIG. 11 is a picture showing a fluorescent image of dish A (not irradiated with a pulse laser without the addition of talaporfin).

FIG. 12 is a picture showing a fluorescent image of dish B (not irradiated with a pulse laser after the lapse of 24 hours from the addition of talaporfin).

FIG. 13 is a picture showing a fluorescent image of dish A (irradiated with a pulse laser for 10 minutes without the addition of talaporfin).

FIG. 14 is a picture showing a fluorescent image of dish B (irradiated with a pulse laser for 10 minutes after the lapse of 1.5 hours from the addition of talaporfin).

### MODE FOR CARRYING OUT THE INVENTION

Hereinafter, the preferred embodiment for carrying out the present technology will be described. Note that the embodiments described below show representative embodiments of the present technology, and thus the scope of the present technology is not narrowly interpreted. The description will be given in the following order.

1. Light Irradiation Method

(1) Pulse Laser

(2) Photosensitive Compound Capable of Generating Singlet Oxygen

(3) Light Irradiation Method

2. Light Irradiation Device

3. Device System for Photodynamic Diagnosis or Photodynamic Therapy

(1) Configuration of Device System for Photodynamic Diagnosis or Photodynamic Therapy

(2) First Imaging Unit

(3) Second Imaging Unit

(4) First Embodiment of Device System for Photodynamic Diagnosis or Photodynamic Therapy

4. System for Specifying a Tumor Site and System for Treating a Tumor

5. Experimental Example of Photodynamic Therapy (PDT)

(1) Material

(2) Experiment 1

(3) Experiment 2

(4) Experiment 3

(5) Experiment 4

### <1. Light Irradiation Method>

In the light irradiation method of the present technology, irradiation of a cell into which a photosensitive compound capable of generating singlet oxygen has been incorporated with a pulse laser having a wavelength within a Soret band is performed.

(1) Pulse Laser

In the present technology, a highly-repeated pulse laser is used. A pulse laser is a pulse light having a short time width of around nanoseconds, picoseconds, or femtoseconds, and can concentrate energy with high repetition within a shorter time width than a simple laser.

The highly-repeated pulse laser used in the present technology is not particularly limited, and a pulse laser with a pulse width of a picosecond level or less can be used. For example, the pulse width is 100 psec or less, more preferably 10 psec or less, and furthermore preferably 1 psec or less, and is preferably a lifetime of the fluorescence generated from a photosensitive compound or shorter. Further, the repetition is preferably 100 MHz or more, and more preferably 1 GHz or less.

If the pulse width is 100 psec or less, the average irradiation energy becomes smaller in a case where the same amount of energy is given, as compared with continuous light. Furthermore, the thermal relaxation time of the proteins existing in a large amount in the living tissues to which energy should not be given is 100 psec or more, therefore, excitation is not repeated, and the thermal influence is hardly given. Moreover, if the repetition is 100 MHz or more, the excitation lifetime of the porphyrin skeleton is around 10 nsec, and the repeated excitation state can be maintained. Since the generation of active oxygen depends on the encounter probability and distance with the oxygen in a ground state, generation efficiency of active oxygen is increased by being kept with the repeated excitation of the porphyrin skeleton, therefore, this is preferred.

The wavelength of the pulse laser used in the present technology is, for example, a wavelength included in a Soret band of a cyclic tetrapyrrole, or the like . Specifically, the wavelength of the pulse laser is 300 nm or more to 500 nm or less. The lower limit of the wavelength is preferably 350 nm or more, more preferably 370 nm or more, and furthermore preferably 395 nm or more. Further, the upper limit of the wavelength is preferably 450 nm or less, more preferably 420 nm, and furthermore preferably 415 nm or less. However, depending on the type of the photosensitive compound as described later, a suitable wavelength can be selected. As the wavelength, for example, a wavelength described in a package insert of each of the photosensitive compounds available on the market can be selected.

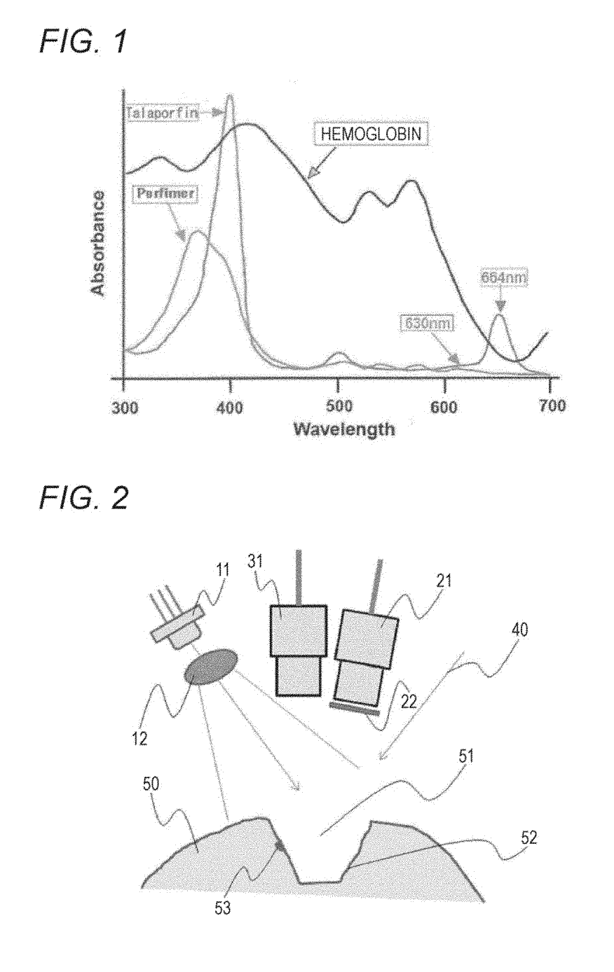

Herein, in FIG. 1, as an example, a graph of absorbance of talaporfin (Laserphyrin (registered trademark)), porfimer (Photofrin (registered trademark)), and hemoglobin is shown.

Both of talaporfin and porfimer have absorbance peaks in the vicinity of the Q band and in the vicinity of the Soret band, and the peak in the vicinity of the Soret band is larger than the peak in the vicinity of the Q band. Therefore, it is considered that there is an advantage in using a short wavelength as in the Soret band for PDD and PDT.

In the conventional PDT, the irradiation is performed with a laser at 664±2 nm if talaporfin is used, and with a laser at 630 nm if porfimer is used. As can be understood from the graph of hemoglobin in FIG. 1, this is considered because the absorbance of hemoglobin in the vicinity of the Q-band is low, and a cyclic tetrapyrrole of the hemoglobin contained in a red blood cell is hardly excited even if being irradiated with light in the vicinity of the Q band. However, in the present technology, by using a pulse laser in a Soret band, the average power contributing to the thermal influence can be suppressed while increasing the depth of invasion with the high peak power. With this arrangement, it is considered that in breast cancer with no mucosal tissue and with the tissue penetration not higher than that in the lungs and brain, tumor can be killed to the extent of the deep part while improving the safety against burn injury.

If irradiation of a cell into which a photosensitive compound capable of generating singlet oxygen has been incorporated with a pulse laser having a wavelength within the Soret band is performed, the photosensitive compound is excited to emit fluorescence, and the tumor site can be easily specified. Further, the tumor can be killed by the produced singlet oxygen (active oxygen).

Note that conventionally, light at a wavelength within a Soret band of a cyclic tetrapyrrole (porphyrin or the like) has been generally used to specify the tumor site by lowering the intensity of light in PDD. However, it has been considered that if the intensity of light is increased to the extent of being used in PDT, a cyclic tetrapyrrole of the hemoglobin contained in a red blood cell is excited and a damage is given to the red blood cell, accordingly, in PDT, it was rare to use the light at a wavelength within the Soret band of a cyclic tetrapyrrole or the like. Therefore, a method of giving an energy amount with low energy per unit area has been desired.

As a specific example of the pulse laser used in the present technology, for example, a laser having the following characteristics can be mentioned.

Crystal: BBO (Castech) Type I phase matching 4×4×0.5 mm theta=29 deg

Crystal length: 0.5 mm

Allowable angle width: 16 mrad (at 0.5 mm)

Light source: Insight (Newport), pulse width 120 fs, repetition 80 MHz

Beam diameter: 1.3 mm (1/e²), φ4 mm in an irradiation unit

Incident wavelength: 810 nm

Condenser lens focal distance: 60 mm (doublet)

Beam converging angle: 10.8 mrad

S-polarized light incidence, a crystal is mounted so that angle phase matching adjustment can be performed with the rotation in the horizontal direction.

### (2) Photosensitive Compound Capable of Generating Singlet Oxygen

As the photosensitive compound (photosensitizer) capable of generating singlet oxygen and used in the present technology, for example, a compound having a cyclic tetrapyrrole can be mentioned. Examples of the cyclic tetrapyrrole include porphyrin, phthalocyanine, corrole, chlorine, bacteriochlorin, and isobacteriochlorin, but are not particularly limited. Further, a metal may be chelated inside these rings.

Such a compound is available as an agent for PDT or an agent for PDD. For example, the following is included:

HPD porfimer (Photofrin II), BPD-MA (Verteporfin/Visudyne), 5-ALA (Levulan), hexaminolevulinate hydrochloride (Hexvix), SnET2 (Photrex), Anecortave acetate (Retaane), 8-methoxypsoralen (Methoxalen), Dihematoporphyrin derivative (Prednisolone), 5-ALA methyl aminolevulinate (Metvix), 5-ALA benxylester (Benzix), talaporfin (Laserphyrin), Diethylene glycol benzoporphyrin (Lemuteporfin), Motexafin Lutetium (Antrin), M-THPC (Foscan), HPPH (Photochlor), Phthalocyanine-4 (Pc4), Silicone phthalocyanine-4 (SiPc4), Lutetium texaphyrin (Lutex), Boronated protoporphyrin (BOPP), Photorex (Rostaporfin), Tookad (Padoporfin), Methyl aminolevulinate (Metvixia), Tin ethyl etiopurpurin (Purlytin), WST11 (Stakel), Aluminum phthalocyanine tetrasulfonate (Photosens), Hypericin, Methylene blue, Toluidine blue, Rose bengal, TH9402, Merocyanine 540, Curcumin.

These compounds have a highly accumulating property to a tumor and are suitable for use in PDD and PDT.

(3) Light Irradiation Method

The light irradiation target of the present technology is preferably a tumor cell. The tumor cell is not particularly limited, and for example, includes cells related to lung cancer, skin cancer (including melanoma), prostate cancer, gastric cancer, uterine cancer, cervical cancer, bladder cancer, esophageal cancer, lymphoma, breast cancer, basal cell carcinoma, brain tumor, laryngeal cancer, tongue cancer, squamous cell carcinoma, and leukemia. The tumor cell is preferably a reachable superficial tumor cell with a pulse laser used in the present technology, and is particularly preferably a tumor cell in the part with relatively a few blood vessels. For example, breast cancer, prostate cancer, and brain tumor cancer are mentioned.

In particular, in breast cancer, the morbidity rate is high in developed countries, early-stage breast cancer that is expected to have a PDT therapeutic effect has an increasing trend, and further breast cancer commonly recur in many cases. PDD and PDT technologies have not been used for breast cancer in the past, but the present technology can be applied to breast cancer, and PDD and PDT can be performed also in breast cancer.

The light irradiation method of the present technology is performed in the following order: firstly, a photosensitive compound capable of generating singlet oxygen is incorporated into a target cell, and then irradiation with the above-described pulse laser is performed.

The photosensitive compound can be prepared into a pharmaceutical preparation for injection, a pharmaceutical preparation for oral administration, or the like.

In the photosensitive compound, for example, in talaporfin (Laserphyrin), if talaporfin is administered to a tumor cell as talaporfin sodium of a pharmaceutical preparation for injection, talaporfin accumulates in a tumor cell. By irradiating with a pulse laser after the lapse of 4 to 6 hours from the administration, fluorescence is emitted from the cell in which talaporfin has accumulated, and the site of the tumor cell is specified. After specifying the site of the tumor cell, the intensity of a pulse laser is appropriately increased, and if the tumor cell is subsequently irradiated with the pulse laser, talaporfin reacts with oxygen to generate singlet oxygen, and the oxidation action gives a thermal damage to the tumor cell, and kills the tumor. Since the photosensitive compound hardly accumulates in normal cells, the damage by the laser is small. Note that in breast cancer or the like, the intensity of the pulse laser and the like can be adjusted so that the thermal damage is not extremely strong and burn injury is not caused.

### <2. Light Irradiation Device>

The light irradiation device of the present technology is equipped with a light irradiation unit for irradiating a cell into which a photosensitive compound capable of generating singlet oxygen has been incorporated with a pulse laser having a wavelength within a Soret band.

In the light irradiation device of the present technology, for example, a light source capable of irradiating a pulse laser having a wavelength within a Soret band of a cyclic tetrapyrrole or the like and preferably a wavelength of 405±10 nm, and further having a pulse width of a picosecond level or less and preferably 100 psec or less can be used as the light irradiation unit.

The light irradiation device is not particularly limited in the present technology, and irradiation can be performed with an average power of 1 mW. It is preferred that depending on the site and the like of the cancer cells to be a tumoricidal target, the irradiation power density, the irradiation energy density, the irradiation time, and the like can be controlled.

### <3. Device System for Photodynamic Diagnosis or Photodynamic Therapy>

The light irradiation system of the present technology has

a light irradiation device equipped with a light irradiation unit for irradiating a cell into which a photosensitive compound capable of generating singlet oxygen has been incorporated with a pulse laser having a wavelength within a Soret band; and

an image acquisition device equipped with a first imaging unit for imaging an image by the fluorescence emitted from a cell into which a photosensitive compound capable of generating singlet oxygen has been incorporated, and a second imaging unit for imaging an image of the cell arbitrarily by natural light.

### (1) Configuration of Device System for Photodynamic Diagnosis or Photodynamic Therapy

In FIG. 2, an example in which components of the device system for photodynamic diagnosis of the present technology are arranged in the vicinity of a surgical field of breast cancer is shown.

Irradiation of a surgical field 50 is performed with a pulse laser from a pulse laser irradiation unit 11 through a lens unit 12 of a pulse laser irradiation device 10. The natural light observation camera that is a second imaging unit 31 images surgical field 50 observed with the light from a lighting for natural light 40. Herein, as lighting for natural light 40, a usually used shadowless lamp can be used, but if a light source capable of being on-off controlled from the present system is used, the work of on-off turning of the shadowless lamp can be omitted.

(2) First Imaging Unit

If the PDD is performed, irradiation of a surgical field is performed with the light that excites the above-described photosensitive compound, and the fluorescence emitted by exciting the photosensitive compound accumulated in the main part is observed with a PDD observation camera that is a first imaging unit 21 (FIG. 2).

Herein, by using a pulse laser light source having a repetition rate faster than the relaxation time of the photosensitive compound (for example, if the relaxation time is a usec level, the repetition rate is MHz order or more) and having a high peak value, the average power can be lowered while sufficiently exciting the photosensitive compound as with a continuous wave (CW) laser. Further, in a case where sufficient fluorescence is obtained for PDD, a light-emitting diode (LED) may be used instead of the pulse laser beam.

In order to clearly observe the fluorescence emitted by the photosensitive compound, in the above-described PDD observation camera, an optical filter 22 for transmitting the fluorescence emitted by the compound and further for cutting off the light that excites the compound can be arranged. Furthermore, optical filter 22 may be a combination of a filter that absorbs the photosensitive compound excitation light and a filter that passes the photosensitive compound fluorescent light. By using such a filter in the PDD observation camera, the fluorescence emitted from a tumor 53 in the stump of a resection area 51 in surgical field 50 is easily captured.

Note that the PDD observation camera has been described as an example here, and even if the PDD observation camera is replaced with a camera for PDT, the similar description is also applied to the camera for PDT.

### (3) Second Imaging Unit

A surgeon wants to locate the tumor site, and the image that the surgeon is looking at during surgery is an image under natural light lighting, therefore, in the present system, second imaging unit 31 (FIG. 2) that images an image of natural light lighting may be provided.

In addition, the image pickup device of the PDD observation camera (first imaging unit 21) is an image pickup device capable of imaging a color image, and if the insertion and removal of optical filter 22 provided in front of the PDD observation camera can be performed at high speed, the cameras can be made into one set. However, if cameras that image the PDD observation image and the natural light observation image, respectively are arranged, it is not required to perform the insertion and removal of optical filter 22 at high speed.

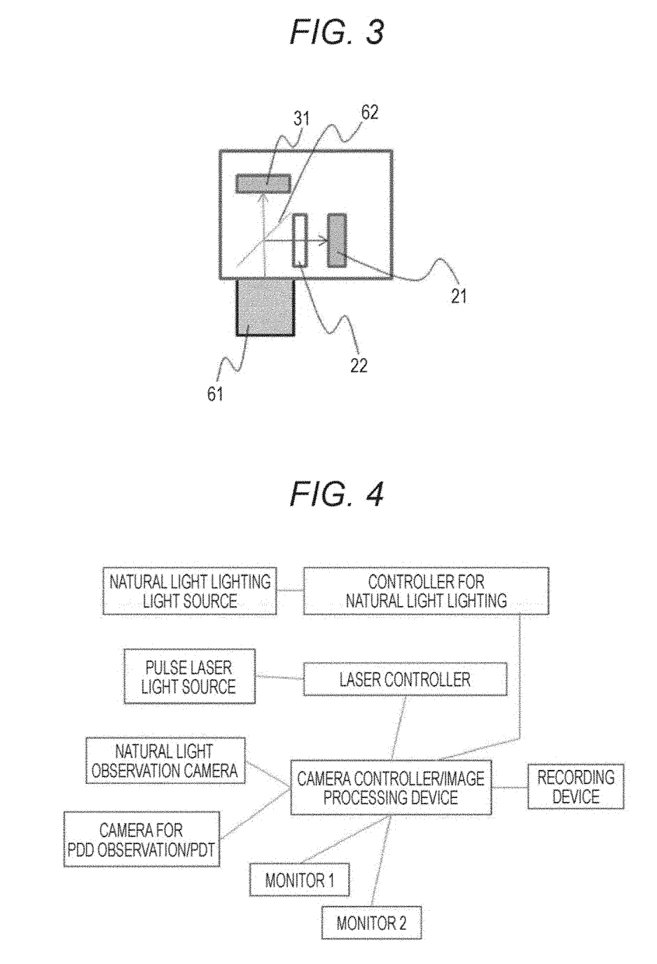

Therefore, in FIG. 3, an example in which the PDD observation camera (first imaging unit 21) and the natural light observation camera (second imaging unit 31) are arranged in one chassis is shown.

The light transmitted through an integrated camera lens unit 61 is split into the light to a PDD observation imager of first imaging unit 21 and the light to a natural light observation imager of second imaging unit 31 by a partial pass filter 6 (beam splitter), and an image is formed in each imaging unit.

### (4) First Embodiment of Device System for Photodynamic Diagnosis or Photodynamic Therapy

FIG. 4 shows a schematic diagram of the first embodiment of the device system for photodynamic diagnosis or photodynamic therapy of the present technology.

Irradiation of a surgical field with each of the natural light and the pulse laser is performed from each of a natural light lighting light source and a pulse laser light source while controlling by a controller for natural light lighting and a controller for a pulse laser. The controller for natural light lighting and the controller for a pulse laser are connected to a camera controller/image processing device.

On the other hand, a natural light observation camera and a camera for PDD observation/PDT are also connected to a camera controller/image processing device, these cameras are controlled by a camera controller, the obtained image is processed in an image processing device, and the data are sent and recorded to a recording device. Further, the image data of the camera controller/image processing device is displayed on monitors 1 and 2.

In these monitors, an image obtained by enlarging the image taken with natural light lighting, and an image (PDD/PDT image) from a camera for PDD observation/PDT that observes the fluorescence emitted with a pulse laser from tumor cells to which a large amount of photosensitive compound has been incorporated can be separately displayed. Moreover, two images can be displayed on one monitor while switching between the two images.

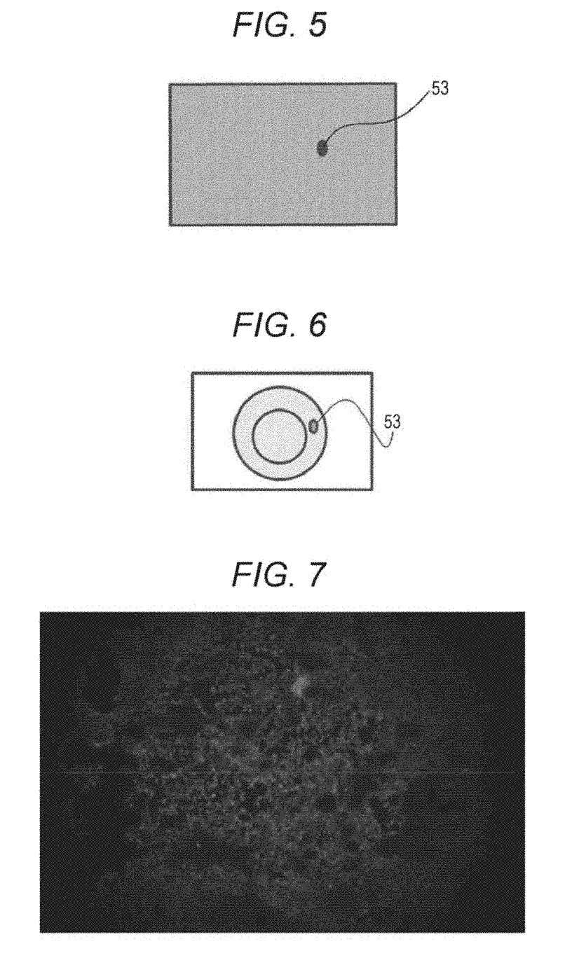

In FIG. 5, an example of an image taken by a camera for PDD observation/PDT is shown. Tumor 53 is observed by fluorescence.

This PDD/PDT image shows only the tumor cells to which photosensitive compound has accumulated, therefore, the surgeon can easily know the presence or absence of tumor cells. However, it is not easy to locate the tumor site.

Accordingly, as shown in FIG. 6, under the calibration condition of photographing positions of the previously adjusted camera for PDD observation/PDT and natural light observation camera, the PDD/PDT image is superimposed on the natural light observation image by matching the positions so that an image (fusion image) can be formed. In this case, it is preferred that the location of tumor 53 (location of the fluorescence emitted by a photosensitive compound) is displayed so that the surgeon can easily understand the location.

In the fusion image as shown in FIG. 6, by inputting the luminance information of the image obtained from the PDD/PDT observation camera to a green channel of color signal of the natural light observation camera, the location of tumor part can be expressed in green that is not the color present in a living body, therefore, the fusion image makes the tumor part (tumor 53) stand out, and as a result, the effect of preventing the positive surgical margin can be enhanced.

In addition, the integrated camera as shown in FIG. 3 has a configuration in which it is difficult for the user to change the photographing positions of the camera for PDD observation/PDT and the natural light observation camera, therefore, by using the integrated camera, the calibration work can be facilitated and the space saving can be achieved.

Further, by recording the image taken in the system into the recording device, the result of the surgery can remain as an image.

In addition, by displaying diagnostic images of mammography, CT, ultrasound, and the like, which have been taken before the surgery, on a monitor during the surgery, the surgeon can easily confirm the location of the tumor part.

Further, by displaying an image taken by a camera for PDD observation/PDT and an image taken by a natural light observation camera on a monitor at the same magnification, or by displaying an image obtained by synthesizing the images taken by the camera for PDD observation/PDT and the natural light observation camera and an image taken by mammography, CT, or a ultrasound diagnostic device on a monitor at the same magnification, the surgeon can compare the diagnostic images with the observation image at the surgery with high accuracy.

Herein, since diagnostic images obtained with mammography, CT, ultrasonic waves, and the like are cross-section information and perspective information, and since images obtained by a natural light observation camera and a camera for PDD observation/PDT are information on a surface of the surgical field, alignment of these two types of information is not generally easy. However, by displaying these images at substantially the same magnification, the surgeon can easily locate the tumor that is present in the diagnostic images. As described above, by displaying the images at substantially the same magnification, the part that is considered to be a tumor area in the diagnostic images and is determined to be excised can be easily found as compared with the case where the images are not displayed at substantially the same magnification or the case where the surgical field is not displayed on a monitor.

On a monitor, the images can also be enlarged and displayed, therefore, it becomes easier to find the tumor part remaining in an enclave state in the surroundings.

Further, in the conventional breast cancer surgery, it is common to mark the target resection area on the breast of a patient with a magic marker pen on the basis of the diagnostic images, and the stereoscopic surgical resection area is regarded as plane perspective information. However, by using this method of the present invention, the information lost by making the stereoscopic information into the plane information can be supplemented.

### <4. System for Specifying Tumor Site and System for Treating Tumor>

The system for specifying a tumor site or system for treating a tumor of the present technology includes an agent containing a photosensitive compound capable of generating singlet oxygen, and a light irradiation device equipped with a light irradiation unit for irradiating a cell into which the photosensitive compound has been incorporated with a pulse laser having a wavelength within a Soret band.

The agent may be a pharmaceutical preparation suitable for arbitrary administration form such as a pharmaceutical preparation for injection, and a pharmaceutical preparation for oral administration. For example, a commercially available drug containing the photosensitizer is suitably used. After the administration, it is preferred to handle the subject in accordance with the description in the package insert of each commercially available drug.

As the pulse laser used in the irradiation from a light irradiation device, a laser having the wavelength and pulse width described above is preferred. The intensity of the pulse laser for PDD and the intensity of the pulse laser for PDT are not particularly distinguished from each other, however, PDD can be performed even if the intensity of the pulse laser is low, but in PDT, the intensity of the pulse laser is set to be high to the extent that tumor can be killed.

The wavelength, pulse width, and the like of a pulse laser for PDD can be changed depending on the drug to be combined. In a case where talaporfin (Laserphyrin) is used for the agent, preferably, a pulse laser having a wavelength of 405±10 nm and a pulse width of 100 psec can be mentioned. In a case where porfimer (Photofrin) is used for the agent, preferably, a pulse laser having a wavelength of 370±10 nm and a pulse width of 100 psec can be mentioned.

Further, the wavelength, pulse width, and the like of a pulse laser for PDT may be the same as the wavelength, pulse width, and the like of the pulse laser for PDD described above, respectively.

### EXAMPLES

### <5. Experimental Example of Photodynamic Therapy (PDT)>

### (1) Material

In vitro experiments of PDT were performed by using the following materials.

Target cells: MCF7 (Human breast adenocarcinoma-derived cell line)

Dish: φ35 mm glass bottom dish (non-coated)

Culture medium: D-MEM-10% FBS-1% P/S-1 mM pyruvate sodium

Photosensitizer: 10 μg/ml talaporfin

Viability assay fluorescence reagent: 10 μg/ml calcein

### (2) Experiment 1

Firstly, dishes A and B in which MCF7 had been cultured in the culture medium described above were prepared. Into the dish A, talaporfin was not added, and the dish A was used as a control. Into the dish B, talaporfin at 10 μg/ml was added. After each dish was incubated for 24 hours, the irradiation with a pulse laser having a wavelength of 405 nm, a pulse width of 120 fs, and an average power of 1 mW (φ4 mm irradiation unit) was performed for 30 minutes. After the irradiation, incubation was performed further for 18 hours, the cultured cells in each dish were washed twice with phosphate-buffered saline (PBS), substitution with 1 ml of calcein at 10 μg/ml was performed, and the cultured cells in each dish were observed.

Results of experiment 1:

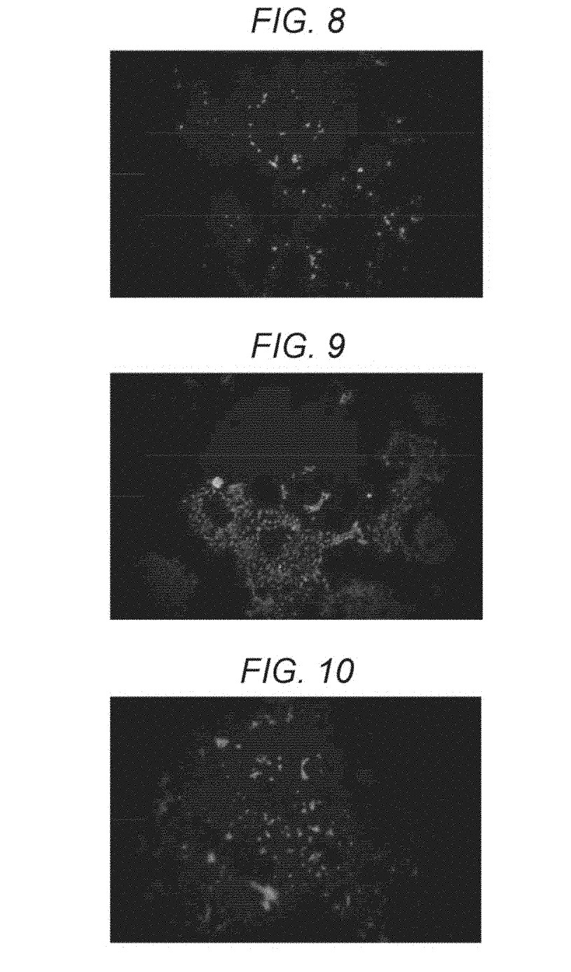

In FIG. 7, a fluorescent image of dish A (irradiated with a pulse laser for 30 minutes without the addition of talaporfin) is shown. In FIG. 8, a fluorescent image of dish B (irradiated with a pulse laser for 30 minutes after the addition of talaporfin) is shown.

It was observed that in FIG. 7, the fluorescence was emitted overall, but in FIG. 8, the fluorescence was scattered. That is, it was suggested that in the dish A, the cultured cells (cells derived from human breast adenocarcinoma) were hardly killed, but in the dish B, the cultured cells were significantly killed as compared with the dish A.

### (3) Experiment 2

In dish A (without the addition of talaporfin) and dish B (with the addition of talaporfin), the cultured cells of each dish were observed in the similar manner as in Experiment 1 except that irradiation of a place different from the place used in the above experiment with a pulse laser was performed for 10 minutes.

### Results of Experiment 2:

In FIG. 9, a fluorescent image of dish A (irradiated with a pulse laser for 10 minutes without the addition of talaporfin) is shown. In FIG. 10, a fluorescent image of dish B (irradiated with a pulse laser for 10 minutes after the addition of talaporfin) is shown.

It was observed that in FIG. 9, the fluorescence was widely and finely emitted, but in FIG. 10, the fluorescence was scattered. That is, in the dish A, the cultured cells (cells derived from human breast adenocarcinoma) were hardly killed, but in the dish B, it was suggested that the cultured cells were significantly killed even with the pulse laser irradiation for 10 minutes, as compared with the dish A.

In addition, it was not able to be confirmed that in the parts where irradiation with a pulse laser had not been performed of the dishes A and B, the cultured cells were killed in both of the dishes A and B.

### (4) Experiment 3

In dish A (without the addition of talaporfin) and dish B (with the addition of talaporfin), the cultured cells of each dish were observed in the similar manner as in Experiment 1 except that irradiation of a place different from the place used in the above experiment with a pulse laser was not performed.

### Results of Experiment 3:

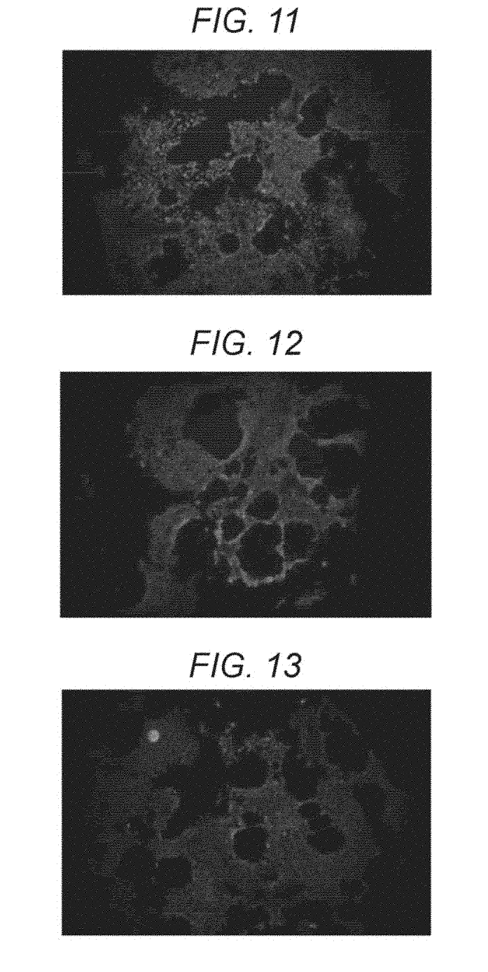

In FIG. 11, a fluorescent image of dish A (not irradiated with a pulse laser without the addition of talaporfin) is shown. In FIG. 12, a fluorescent image of dish B (not irradiated with a pulse laser with the addition of talaporfin) is shown.

In both of FIGS. 11 and 12, the cells were alive without the laser irradiation, therefore, it was confirmed that the cells were not killed only with the addition of talaporfin.

### (5) Experiment 4

In dish B (with the addition of talaporfin), after the addition of taraporfin, incubation was performed for 1.5 hours, and the cultured cells of each dish were observed in the similar manner as in Experiment 1 except that irradiation of a place different from the place used in the above experiment in dish A (without the addition of talaporfin) and dish B with a pulse laser was performed for 10 minutes.

### Results of Experiment 4:

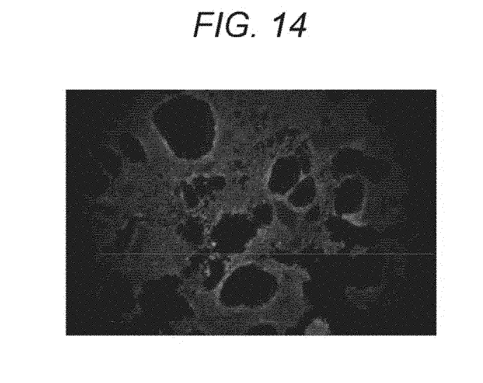

In FIG. 13, a fluorescent image of dish A (irradiated with a pulse laser for 10 minutes without the addition of talaporfin) is shown. In FIG. 14, a fluorescent image of dish B (irradiated with a pulse laser for 10 minutes with the addition of talaporfin (1.5 hours)) is shown.

In both of FIGS. 13 and 14, it was confirmed that the cells were alive. From the result of the dish B, it was suggested that even if talaporfin was added, and even if irradiation with a pulse laser was performed, the cells were alive if the cells had not fully incorporated the talaporfin.

From the above, it was able to be confirmed that if a photosensitive compound is incorporated into cells, and irradiation of the cells with a pulse laser at 405 nm is performed, a photodynamic therapy effect is obtained. Further, it was also able to be confirmed that the tumoricidal effect was not observed by the irradiation only with a pulse laser without incorporating the photosensitive compound into the cells, the tumoricidal effect was not observed also only by incorporating the photosensitive compound into the cells without irradiation with a pulse laser, and the tumoricidal effect was not observed even if the irradiation with a pulse laser had been performed without incorporating the photosensitive compound sufficiently into the cells (if the time of incorporation was short).

In addition, the present technology can also adopt the following constitution.

[1] A light irradiation method, including:

irradiating a cell into which a photosensitive compound capable of generating singlet oxygen has been incorporated with a pulse laser having a wavelength within a Soret band.

[2] The light irradiation method according to [1], in which

a pulse width of the pulse laser is 100 psec or less.

[3] The light irradiation method according to [1] or [2], in which the wavelength is 405±10 nm.

[4] The light irradiation method according to any one of [1] to [3], in which

the photosensitive compound capable of generating singlet oxygen is an agent for photodynamic therapy (PDT) or an agent for photodynamic diagnosis (PDD).

[5] The light irradiation method according to any one of [1] to [4], in which

the photosensitive compound capable of generating singlet oxygen is a compound having a cyclic tetrapyrrole.

[6] The light irradiation method according to [5], in which

the compound having a cyclic tetrapyrrole is a compound generated by metabolism.

[7] The light irradiation method according to [5] or [6], in which

the cyclic tetrapyrrole is porphyrin.

[8] The light irradiation method according to any one of [1] to [7], in which

the cell is a tumor cell.

[9] The light irradiation method any one of [1] to [8], in which

the cell is a breast cancer cell.

[10] A light irradiation device, including:

a light irradiation unit for irradiating a cell into which a photosensitive compound capable of generating singlet oxygen has been incorporated with a pulse laser having a wavelength within a Soret band.

[11] A device system for photodynamic diagnosis or photodynamic therapy, including:

a light irradiation device equipped with a light irradiation unit for irradiating a cell into which a photosensitive compound capable of generating singlet oxygen has been incorporated with a pulse laser having a wavelength within a Soret band; and

an image acquisition device equipped with a first imaging unit for imaging an image by fluorescence emitted from a cell into which a photosensitive compound capable of generating singlet oxygen has been incorporated.

[12] The device system for photodynamic diagnosis or photodynamic therapy according to [11], in which

the image acquisition device is equipped with a second imaging unit for imaging an image of the cell by natural light.

[13] A system for specifying a tumor site, including:

an agent containing a photosensitive compound capable of generating singlet oxygen; and

a light irradiation device equipped with a light irradiation unit for irradiating a cell into which the photosensitive compound has been incorporated with a pulse laser having a wavelength within a Soret band.

[14] A system for treating a tumor, including:

an agent containing a photosensitive compound capable of generating singlet oxygen; and

a light irradiation device equipped with a light irradiation unit for irradiating a cell into which the photosensitive compound has been incorporated with a pulse laser having a wavelength within a Soret band.

### REFERENCE SIGNS LIST

10 Pulse laser irradiation device
11 Pulse laser irradiation unit
12 Lens unit
21 First imaging unit
22 Optical filter
31 Second imaging unit
40 Lighting for natural light
50 Surgical field
51 Resection area
52 Stump
53 Tumor
61 Integrated camera lens unit

1. A light irradiation method, comprising:
irradiating a cell into which a photosensitive compound capable of generating singlet oxygen has been incorporated with a pulse laser having a wavelength within a Soret band.

2. The light irradiation method according to claim 1, wherein
a pulse width of the pulse laser is 100 psec or less.

3. The light irradiation method according to claim 1, wherein
the wavelength is 405±10 nm.

4. The light irradiation method according to claim 1, wherein
the photosensitive compound capable of generating singlet oxygen is an agent for photodynamic therapy (PDT) or an agent for photodynamic diagnosis (PDD).

5. The light irradiation method according to claim 1, wherein
the photosensitive compound capable of generating singlet oxygen is a compound having a cyclic tetrapyrrole.

6. The light irradiation method according to claim 5, wherein
the compound having a cyclic tetrapyrrole is a compound generated by metabolism.

7. The light irradiation method according to claim 5, wherein
the cyclic tetrapyrrole is porphyrin.

8. The light irradiation method according to claim 1, wherein
the cell is a tumor cell.

9. The light irradiation method according to claim 1, wherein
the cell is a breast cancer cell.

10. A light irradiation device, comprising:
a light irradiation unit for irradiating a cell into which a photosensitive compound capable of generating singlet oxygen has been incorporated with a pulse laser having a wavelength within a Soret band.

11. A device system for photodynamic diagnosis or photodynamic therapy, comprising:
a light irradiation device equipped with a light irradiation unit for irradiating a cell into which a photosensitive compound capable of generating singlet oxygen has been incorporated with a pulse laser having a wavelength within a Soret band; and
an image acquisition device equipped with a first imaging unit for imaging an image by fluorescence emitted from a cell into which a photosensitive compound capable of generating singlet oxygen has been incorporated.

12. The device system for photodynamic diagnosis or photodynamic therapy according to claim 11, wherein
the image acquisition device is equipped with a second imaging unit for imaging an image of the cell by natural light.

13. A system for specifying a tumor site, comprising:
an agent containing a photosensitive compound capable of generating singlet oxygen; and
a light irradiation device equipped with a light irradiation unit for irradiating a cell into which the photosensitive compound has been incorporated with a pulse laser having a wavelength within a Soret band.

14. A system for treating a tumor, comprising:
an agent containing a photosensitive compound capable of generating singlet oxygen; and
a light irradiation device equipped with a light irradiation unit for irradiating a cell into which the photosensitive compound has been incorporated with a pulse laser having a wavelength within a Soret band.
